(19) United States
(12) Patent Application Publication
(10) Pub. No.: US 2024/0261556 A1
(43) Pub. Date: Aug. 8, 2024

(54) MICROCATHETER MANIFOLD ADAPTER AND ASSOCIATED SYSTEMS AND METHODS

(71) Applicant: BOSTON SCIENTIFIC SCIMED, INC., MAPLE GROVE, MN (US)

(72) Inventors: Robert M. Wold, Maple Lake, MN (US); Justin Robert Alt, Minneapolis, MN (US); Jordon Lewis, Ramsey, MN (US)

(73) Assignee: BOSTON SCIENTIFIC SCIMED, INC., MAPLE GROVE, MN (US)

(21) Appl. No.: 18/429,685

(22) Filed: Feb. 1, 2024

Related U.S. Application Data
(60) Provisional application No. 63/442,905, filed on Feb. 2, 2023.

(51) Int. Cl. A61M 39/10; A61M 25/00

(52) U.S. Cl. CPC A61M 39/10; A61M 25/0097; A61M 2025/0042; A61M 2039/1077

(57) ABSTRACT

An adaptor for modifying pre-existing delivery system to improve delivery of an injectable treatment material through the delivery system. The delivery system is a pre-existing delivery system formed separately and without regard to the manufacture of the adaptor. The delivery system includes an administration set and a catheter set coupled together in fluid communication via a manifold. The injectable treatment material may be in the form of microspheres or microbeads which may become held up within the void space of the pre-existing manifold of the delivery system. The adaptor reconfigures the interior of the manifold to facilitate complete flow of the injectable treatment material therethrough without getting hung up or otherwise caught within the manifold and not delivered to a patient.

### CROSS-REFERENCE TO RELATED APPLICATIONS

The present application claims the benefit of priority under 35 U.S.C. § 119 to U.S. Provisional Application No. 63/442,905, filed Feb. 2, 2023, all of the disclosure of which is hereby incorporated herein by reference in its entirety for all purposes. Any and all priority claims identified in the Application Data Sheet, or any correction thereto, are hereby incorporated by reference under 37 C.F.R. § 1.57.

### FIELD

The present disclosure relates generally to the field of devices, systems, and methods for delivering treatment materials to a patient's body. In particular, the present disclosure relates to medical devices, systems, and methods for adapting existing injectable treatment material delivery devices, systems, and methods to improve delivery of injectable treatment materials therethrough. More particularly, the present disclosure relates to medical devices, systems, and methods for adapting existing treatment material delivery devices, systems, and methods to effectively delivery injectable treatment materials in the form of microspheres, microbeads, etc.

### BACKGROUND

Various devices, systems, and methods exist to deliver injectable treatment materials to a patient's body. The injectable treatment material may be contained within a vial. Any of a variety of administration sets may be used to access the injectable material from within the vial to deliver the injectable material to the patient. Typically, the administration set includes a flexible tubular element fluidly coupled with the vial at one end, and having a fitting/connector at the other end for coupling with a catheter or microcatheter which accesses the patient's body to deliver the injectable treatment material thereto. The catheter or microcatheter typically includes a manifold at one end configured to be coupled with the administration set. The other end of the microcatheter typically is configured to be inserted into the patient's body to deliver the injectable treatment material into the patient's body. The manifold typically has a fitting/connector configured to be coupled with the administration set's fitting/connector. Various challenges are presented with certain injectable treatment materials being able to pass fully through the manifold and into the catheter or microcatheter. For instance, some injectable treatment materials have properties which cause the injectable treatment material to get stuck within the manifold. Such issue becomes significant when a low dose of the injectable treatment material is administered and greater than about 5% of the injectable treatment material is not transmitted through the manifold and into the patient's body. Solutions to these and other challenges would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an adaptor is configured to modify a separately manufactured delivery system through which an injectable treatment material is delivered to a patient's body. In some aspects, the adaptor has an adaptor body extending from a proximal end to a distal end, with a lumen defined through the adaptor body, extending between the proximal end and the distal end of the adaptor body. In some aspects, the adaptor is sized, shaped, configured, and/or dimensioned to fit within a lumen defined through a manifold of the delivery system to reduce the dimensions of the manifold lumen to facilitate flow of the injectable treatment material therethrough from a proximal end of the manifold to a distal end of the manifold. In some aspects, the proximal end of the adaptor is configured to mate with a male fitting of an administration set configured to deliver an injectable treatment material through the manifold; and the distal end of the adaptor is configured to be positioned within the manifold lumen upstream of the proximal end of a catheter within the manifold lumen.

In some embodiments, the adaptor lumen includes a tapered segment. In some embodiments, the tapered segment is adjacent the proximal end of the adaptor body. In some embodiments, the adaptor lumen has a constant-diameter segment extending from the tapered segment to the distal end of the adaptor body. In some embodiments, a tubular element extends within the constant-diameter segment of the adaptor lumen. In some embodiments, the adaptor body is formed of a pliable material and the tubular element is formed of a material more rigid than the adaptor body material. In some embodiments, the adaptor body is molded over the rigid tubular element.

In some embodiments, the proximal end of the adaptor is configured to mate against the face of a fitting of an administration set upstream of the manifold of the delivery system, or to accept a male fitting of the administration set therein.

In some embodiments, the proximal end of the adaptor is configured to mate against the face of a fitting of a line set upstream of the manifold of the delivery system, or to accept a male fitting of the line set therein.

In accordance with various principles of the present disclosure, an adaptor is configured to modify a manifold through which an injectable treatment material flows for delivery to a patient's body. In some aspects, the adaptor has an adaptor body extending from a proximal end to a distal end, and a lumen is defined through the adaptor body, extending between the proximal end and the distal end of the adaptor body. In some aspects, the adaptor is sized, shaped, configured, and/or dimensioned to be inserted into a lumen defined through the manifold to reduce the dimensions of the manifold lumen to facilitate flow of the injectable treatment material therethrough from a proximal end of the manifold to a distal end of the manifold. In some aspects, the adaptor is at least partially formed of a pliable material to conform to the manifold lumen to fill space within the manifold lumen. In some aspects, the adaptor is sized, shaped, configured, and/or dimensioned and formed of a material so that the adaptor body maintains patency of the adaptor lumen when positioned within a manifold.

In some embodiments, the adaptor lumen includes a tapered segment. In some embodiments, the tapered segment is adjacent the proximal end of the adaptor body. In some embodiments, the adaptor lumen has a constant-diameter segment extending from the tapered segment to the distal end of the adaptor body. In some embodiments, a tubular element extends within the constant-diameter segment of the adaptor lumen. In some embodiments, the adaptor body is formed of a pliable material and the tubular element is formed of a material more rigid than the adaptor body material. In some embodiments, the adaptor body is molded over the rigid tubular element.

In some embodiments, a tubular element extends within the adaptor body lumen. In some embodiments, the adaptor body is formed of a pliable material and the tubular element is formed of a material more rigid than the adaptor body material. In some embodiments, the adaptor body is molded over the rigid tubular element.

In some embodiments, the adaptor lumen has a constant diameter section. In some embodiments, a tubular element extends through the constant diameter section of the adaptor lumen. In some embodiments, the adaptor body is formed of a pliable material and the tubular element is formed of a material more rigid than the adaptor body material. In some embodiments, the adaptor body is molded over the rigid tubular element.

In some embodiments, the proximal end of the adaptor is configured to mate against the face of a fitting of a line set upstream of the manifold of the delivery system, or to accept a male fitting of the line set therein.

In accordance with various principles of the present disclosure, method of reconfiguring a system for delivering an injectable treatment material includes inserting a pliable adaptor into the lumen of a pre-existing manifold formed separately and independently of the adaptor; and fitting the adaptor within the manifold lumen to occupy space therein to reduce the volume within the manifold lumen to facilitate flow of the injectable treatment material through the manifold and into a patient's body.

In some aspects, the method further includes providing a tubular element within the adaptor lumen to maintain patency of the adaptor lumen to facilitate flow of the injectable treatment material therethrough.

In some aspects, the method further includes mating a male fitting of a line set with respect to a proximal end of the adaptor. In some aspects, the method further includes inserting the male fitting of the line set into the adaptor lumen within the manifold. In some aspects, the method further includes pushing the adaptor into the manifold with the male fitting of the line set.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of a delivery system with an example of an embodiment of an adaptor formed in accordance with aspects of the present disclosure

FIG. 2 illustrates an exploded perspective view of a delivery system and adaptor as illustrated in FIG. 1.

FIG. 3A illustrates a perspective view of an example of an embodiment of an adaptor formed in accordance with various principles of the present disclosure.

FIG. 3B illustrates a cross-sectional view along line IIIB-IIIB in FIG. 3A.

FIG. 4A illustrates a perspective view of an example of an embodiment of an adaptor formed in accordance with various principles of the present disclosure.

FIG. 4B illustrates a cross-sectional view along line IVB-IVB in FIG. 4A.

FIG. 5A illustrates a perspective view of an example of an embodiment of an adaptor formed in accordance with various principles of the present disclosure.

FIG. 5B illustrates a cross-sectional view along line VB-VB in FIG. 5A.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

The present disclosure describes devices, systems, and methods for delivering injectable treatment materials into a patient's body. It will be appreciated that reference herein is made to “injectable treatment material” or “injectables” for the sake of convenience, and without intent to limit, to refer to treatment materials in an injectable form, including, without limitation, pharmaceuticals, diagnostics (including, without limitation, imaging materials), therapeutic devices (including, without limitation, particles, beads, spheres, etc.), etc., and including solid particles carried by a fluid medium and/or fluids carried by a fluid medium. The injectable material may be a liquid, a solid (e.g., a dry solid, such as microspheres, typically suspended/carried in a fluid), or combinations thereof (e.g., microspheres in deionized water, microspheres with a fluoroscopic contrast medium, etc.). The injectable material may be used for diagnostics, imaging, treatment, therapy, etc., without limitation, the present disclosure not being limited in this context. Moreover, the injectable material may be in any of a variety of desired or indicated forms, such as a fluid (e.g., a typical pharmaceutical form), a gel, or a solid (e.g., solid particles such as beads, microbeads, microspheres, or other particles, such as known for therapeutical use, etc., typically suspended/carried in a fluid). In some instances, the injectable treatment materials may be beads or microbeads or microspheres (such terms may be used interchangeably herein, reference generally being made to microspheres for the sake of convenience and without intent to limit), typically suspended in a fluid medium.

The delivery devices, systems, and methods include administration sets (which may also be referenced as line sets or tubing sets without intent to limit) coupling a vessel containing the injectable treatment material with a catheter configured to deliver the injectable treatment material into the patient's body. The injectable treatment material vessel may be a vial, syringe, etc., the present disclosure not being limited in this regard. Typically, the catheter includes a manifold with a connector, and the administration set includes a connector couplable with the manifold's connector. Additionally, a microcatheter (e.g., having an inner diameter of approximately 0.40-1.30 mm) is typically used to deliver microspheres to a patient's body. However, reference to a “microcatheter” herein need not be so limited, and should be understood to include catheters, neurocatheters, etc., the present disclosure not being limited by the size, dimension, etc., of the catheter.

In some instances, the injectable treatment materials may be radioactive medical injectable treatment materials (e.g., radiopharmaceuticals, radiotherapeutics, radiodiagnostics, radioimaging, tracers, etc.), although the disclosure is not limited in this context. For instance, the injectable treatment materials to be delivered with devices, systems, and methods of the present disclosure may be beads or microbeads or microspheres such as irradiated beads or microbeads or microspheres. An example of a radioactive medical injectable treatment material is TheraSphere™ Y-90 Glass Microspheres (glass microspheres infused with Yttrium, particularly Y-90), sold by Boston Scientific Corporation, for cancer treatment (radiotherapy). TheraSphere™ Microspheres may be delivered via microcatheter to a tumor within the patient's body, and collect in the tumor microvessels for precise, localized tumor ablation. Additionally or alternatively, another radioactive medical injectable material may be provided for imaging and diagnostic purposes. More particularly, microspheres, which may be formed of a suitable material such as glass, may be infused or labelled or coated with radioactive materials, such as Technetium, particularly Tc-99m. The Tc-99m labelled microspheres have substantially the same diameters (average diameters of approximately 25 μm) and substantially the same flow characteristics as the TheraSphere™ Microspheres, and thus may function as a surrogate or proxy for the TheraSphere™ Microspheres. Therefore, the Tc-99m microspheres can be used for imaging and diagnostics before injection of TheraSphere™ microspheres which have a significantly longer half-life (Y-90 has a half-life of 64 hours in contrast with Tc-99m, which has a half-life of 6 hours). It will be appreciated that surface textures on the components of devices and systems formed in accordance with various principles of the present disclosure preferably have structures smaller than the diameter of the particles being passed therethrough to ensure those particles do not “stick” to the components of the devices and systems.

Microspheres such as TheraSphere™ microspheres emit high intensity β-radiation in low doses, for example, doses as small as 1 GBQ per 15 ml of a carrier such as water, or even as small as 0.5 GBQ per 15 ml of a carrier, such as loaded in a vial with up to 0.6 ml of pyrogen-free water delivered with 20 cc of saline via a syringe. When the microspheres are injected into the microcatheter in low doses and at low flow rates, it is critical to the patient that the full dose is administered. However, various aspects of current delivery systems and components thereof may interfere with delivery of a full dose. For instance, microspheres may coagulate in the system, such as along a hub along the system coupling components thereof. For instance, the vial containing the injectable treatment material is typically fluidly coupled to an administration set which, in turn, is typically connected via a manifold to a microcatheter for administration into the patient's body. There may be a void space between the administration set connector and the inner lumen of the microcatheter. More particularly, there may be a void space within the manifold fluidly coupled between the administration set and the microcatheter. As the microspheres travel through the manifold of the microcatheter, the microspheres may stick to the side wall of the manifold, preventing the full dose of microspheres getting to the disease site. Repeated tapping to release the microspheres may unnecessarily expose the medical professional to radiation while tapping, due to the proximity and lack of shielding between the active microspheres and the medical professional. Moreover, such tapping process is generally an uncontrolled, variable process which not only adds strain to the medical professional, but also may produce inconsistent results, which may impact the delivery efficiency and microcatheter dwell time in vivo.

Additionally, up to 10 flushing cycles may be performed to get the microspheres to the disease site. For glioma, less flushing would be desired. Reducing the number of flushes needed to clear the microspheres out of the vial and the delivery system (including the administration set, the manifold, and the microcatheter) reduces patient risk in several ways. First, reducing the number of flushes reduces the risk of injecting too much fluid too fast, which may increase risk of reflux near the catheter tip. Reflux of the microspheres may result in the delivery of therapy to a non-affected region, which is a potential patient risk. Additionally, injecting too much fluid too fast may result in reaching tissue capacitance or edema. Second, reducing the number of flushes will expedite the procedure time, reducing catheter dwell time in vivo, resulting in a reduction in potential patient risk. Finally, reducing the number of flushes will improve the user experience of the system by reducing the amount of physical effort required, reducing mental burden on the user, and/or reducing overall time of the procedure.

In accordance with various principles of the present disclosure, an adaptor separate and separately formed from a pre-existing, pre-formed, separately-formed delivery system is configured to interface with the delivery system to facilitate complete flow of the injectable treatment material through the delivery system and into the patient. As used herein, descriptors such as “pre-existing”, “pre-formed”, “separately-formed”, “separately manufactured”, etc., are intended to refer to an already-made, such as a commercially available, off-the-shelf, device or system which has been formed such as without reference to a particular injectable treatment material such as the microspheres described herein. Adaptors such as described herein are separately and independently formed in accordance with various of the present disclosure to modify various characteristics of an already-made device or system, In this instance, a “pre-formed” delivery system is a delivery system which has been formed separately and independently of the adaptor and has not necessarily been formed for the purpose for which the adaptor has been formed. The adaptor has a lumen in fluid communication with the delivery lumen of the administration set as well as the lumen of the microcatheter of the delivery system through which the injectable treatment material is delivered into the patient's body. In some aspects, the adaptor is configured to be positioned within a manifold coupled between an administration set and a catheter. The manifold is configured to interface with the administration set and/or the catheter to seal connections therebetween to ensure complete flow of the injectable treatment material therethrough. In some aspects, the adaptor interfaces with a male fitting of an administration set to seal the administration set's fitting. In some aspects, the adaptor has a tapered inner diameter that funnels the injectable treatment material to the microcatheter. The exterior form of the adaptor may be configured to prevent the injectable treatment material from filling the manifold's interior void space. The adaptor may be configured so that it will work in a vertical position as well as horizontal position, ensuring the beads are being delivered to the disease site regardless of how the delivery system is positioned by the medical professional during use. In some embodiments, a tubular insert (e.g., a tubular metal insert) is provided within at least a portion of the manifold to provide column strength to the distal end of the adaptor lumen.

It will be appreciated that an adaptor formed in accordance with various principles of the present disclosure minimizes flushing which may be required, thereby reducing the catheter dwell time in-vivo, reducing the overall procedure time, and reducing the risk of inconsistent microsphere delivery, microsphere reflux, tissue capacitance, edema, etc.

Moreover, it will be appreciated that an adaptor formed in accordance with various principles of the present disclosure eliminates the need for tapping components of a delivery system to ensure that the injectable treatment material (e.g., beads) are not hung up in one or more components of the delivery system. For example, placement of an adaptor formed in accordance with various principles of the present disclosure within the manifold of a pre-existing delivery system facilitates flow of an injectable treatment material such that the medical professional administering the injectable treatment material no longer would have to tap the manifold (e.g., with a separate utensil) as the dose is being delivered to the disease site. Use of an adaptor formed in accordance with various principles of the present disclosure thus reduces the risk of increased radiation exposure to both the medical professional and to the patient, reduces catheter dwell time in-vivo, reduces overall procedure time, and reduces the risk of inconsistent microsphere delivery. An adaptor formed in accordance with various principles of the present disclosure is adaptable to any manifold, as the adaptor is formed to meet the existing specifications of the manifold to suit the characteristics of the injectable treatment material to flow therethrough. Moreover, an adaptor formed in accordance with various principles of the present disclosure can be adapted to any microcatheter, catheter, etc. Even if the exterior profile of the adaptor is catheter dependent (if inserted into the same manifold lumen into which the catheter is inserted), but the inner diameters and profile of the adaptor will ensure proper and complete delivery of the injectable treatment material.

Various embodiments of adaptors for devices and systems for delivering injectable treatment materials, and associated methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments of adaptors formed in accordance with various principles of the present disclosure are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

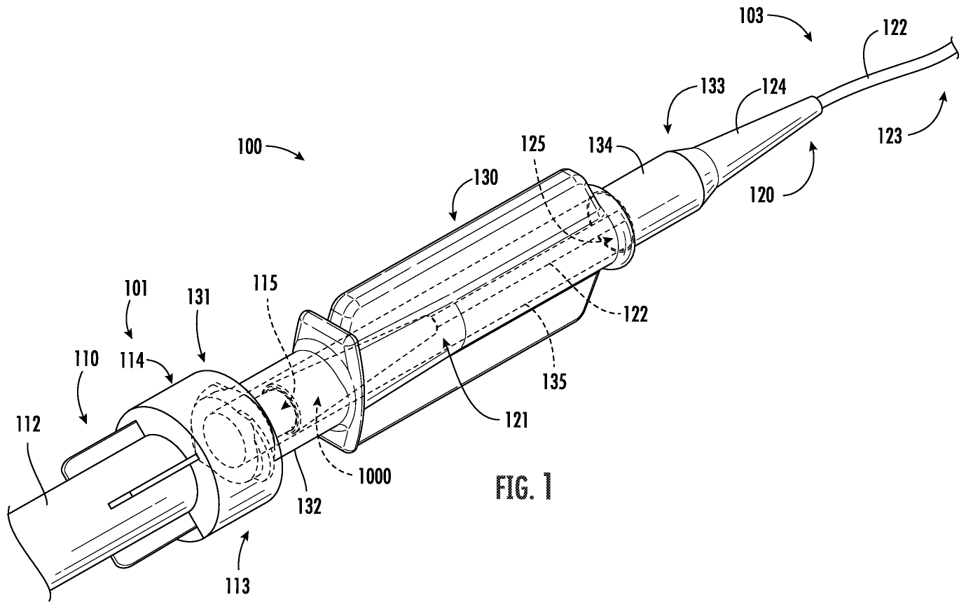

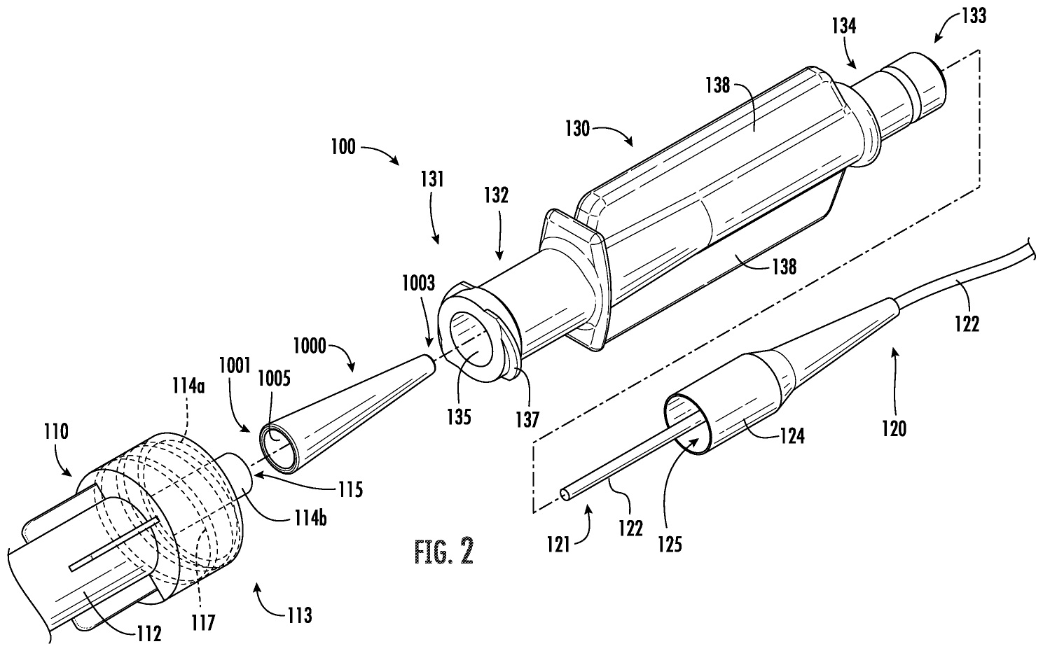

An injectable treatment material delivery system 100 formed in accordance with various principles of the present disclosure is illustrated in FIG. 1 and FIG. 2 as including an administration set 110 at a proximal end 101 thereof, and a microcatheter set 120 at a distal end 103 thereof. The proximal end of the administration set 110 is coupled to a vial containing an injectable treatment material. The proximal end of the administration set 110, the vial, and the injectable treatment material may be in any configuration known to those of ordinary skill in the art, such as depending on the type of injectable treatment material to be delivered by the injectable treatment material delivery system 100, illustration thereof therefore not being necessary for a complete understanding of such elements by those of ordinary skill in the art.

The illustrated examples of embodiments of an administration set 110 and a microcatheter set 120 are coupled together with an example of an embodiment of a manifold 130. More particularly, the distal end 113 of the administration set 110 is coupled with the proximal end 131 of the manifold 130, and the proximal end 121 of the microcatheter set 120 is coupled with the distal end 133 of the manifold 130. Such arrangement places a lumen 115 defined within and through the tubular element 112 of the administration set 110 in fluid communication with a lumen 125 defined within and through the microcatheter 122 of the microcatheter set 120 via a lumen 135 defined within and through the manifold 130. The distal end of the microcatheter 122 may be configured for insertion into a patient for delivery of the injectable treatment material into the patient's body in a manner known to those of ordinary skill in the art. Illustration of the distal end of the microcatheter 122 is thus considered unnecessary for a complete understanding of such element by those of ordinary skill in the art.

As illustrated in further detail in FIG. 2, the administration set 110 includes a fitting 114 extending distally from the distal end 113 of the administration set 110. It will be appreciated that terms such as fitting, connector, coupling, coupler, adaptor, etc., may be used interchangeably herein without intent to limit unless otherwise indicated. The administration set fitting 114 is configured to be coupled with a corresponding fitting 132 extending proximally from the proximal end 131 of the manifold 130. In the illustrated example of an embodiment, a female portion 114a of the administration set fitting 114 is configured to fit over the manifold fitting 132, and a male portion 114b of the administration set fitting 114 is configured to fit into the manifold fitting 132 to place the administration set lumen 115 in fluid communication with the manifold lumen 135. Optionally, the administration set female portion 114a and the manifold fitting 132 have interengaging configurations to hold these elements in place with respect to each other. In the example of an embodiment illustrated in FIG. 1 and FIG. 2, the administration set female portion 114a and the manifold fitting 132 have interengaging respective threads 117, 137. However, the present disclosure is not limited in this regard, and encompasses other interengaging configurations known to those of ordinary skill in the art.

In the example of an embodiment of a microcatheter set 120 illustrated in FIG. 1 and FIG. 2, the microcatheter 122 of the microcatheter set 120 may be coupled with the manifold 130 via a strain relief 124 extending along a proximal portion of the microcatheter 122. The strain relief 124 may be extended onto a distal fitting 134 of the manifold 130 extending distally from the distal end 133 of the manifold 130. The microcatheter 122 may extend proximally from the strain relief 124 further into the manifold lumen 135 to be in closer communication with the lumen 115 defined within the administration set 110.

Optionally, the manifold 130 includes one or more extensions 138, such as wings, extending radially outwardly from the outer surface of the manifold 130 to facilitate gripping thereof. The extensions 138 may extend sufficiently radially outwardly from, as well as longitudinally along, the manifold 130 to facilitate manipulation of the manifold 130, such as rotation of the manifold 130 relative to at least the administration set 110 to couple the manifold 130 and the administration set 110 together.

The present disclosure relates to devices, systems, and methods for modifying a pre-existing, pre-formed, separately-formed, separately manufactured, etc., injectable treatment material delivery systems, such as the above-described system, after the manufacture of such injectable treatment material delivery system. More particularly, the devices, systems, and methods of the present disclosure improve fluid flow through pre-existing, separately-formed injectable treatment material delivery systems such as described above. The manufacture of adaptor devices and systems formed in accordance with various principles of the present disclosure thus need not be dictated by manufacturing requirements of the injectable treatment material delivery system with which the adaptor devices and systems are to be used. As such, adaptor devices and systems may be formed in accordance with various principles of the present disclosure with increased options for improving fluid flow within the manifold of a pre-existing injectable treatment material delivery system to accommodate the requirements of a particular injectable treatment material. The size, shape, configuration, and/or dimensions of the devices and/or systems formed in accordance with various principles of the present disclosure may thus be selected to suit the requirements of the injectable treatment material delivery system and the injectable treatment material to be delivered therethrough. For instance, the size, shape, configuration, and/or dimensions of devices and/or systems formed in accordance with various principles of the present disclosure may be selected in view of the nature, properties, characteristics, etc., of the injectable treatment material to be delivered by the injectable treatment material delivery system with which devices and/or systems of the present disclosure are to be used. For instance, the materials of components of devices and systems formed in accordance with various principles of the present disclosure may be specifically selected to withstand the radiation exposure of radioactive injectable treatment materials passing therethrough.

In accordance with various principles of the present disclosure, a separately formed adaptor 1000 is configured for insertion into the lumen 135 of the manifold 130 of an injectable treatment material delivery system 100 such as described above. The adaptor 1000 has an adaptor body extending from a proximal end 1001 to a distal end 1003 configured to fit within the manifold lumen 135 to adapt a pre-existing or pre-formed manifold 130 for delivery of an injectable treatment material requiring modified flow characteristics from those resulting from flow through at least the manifold 130 as formed. For instance, an adaptor 1000 formed in accordance with various principles of the present disclosure may reduce the size of a manifold lumen 135 to facilitate flow of microbeads or other injectable treatment material, such as described above, to improve delivery of the injectable treatment material to the patient from assistance of a reduction in the diameter of the flow path from the administration set 110 to the microcatheter set 120.

An example of an embodiment of an adaptor 1000 formed in accordance with various principles of the present disclosure is illustrated in FIG. 1 and FIG. 2 as extending within the manifold lumen 135. The proximal end 1001 of the illustrated example of an embodiment of an adaptor 1000 is configured to receive the male portion 114b of the administration set fitting 114. For instance, a distal portion of the male portion 114b of the administration set fitting 114 may be extended into the adaptor lumen 1005. The distal end 1003 of the adaptor 1000 is inserted further into the manifold lumen 135 so that the body of the adaptor 1000 occupies space within the manifold lumen 135 to reduce the dimensions of the manifold lumen 135 and thereby facilitate flow of injectable treatment material, such as microspheres, therethrough. Reduction of the volume within the manifold lumen 135 has been found to reduce the amount of injectable treatment material such a microspheres getting caught or trapped or otherwise preventing the microspheres from hanging up within the void of the manifold lumen 135. The proximal end 121 of the microcatheter 122 is inserted into the distal end 133 of the manifold 130 to be in proximity with the distal end 1003 of the adaptor 1000. Optionally, a step or shoulder is provided within the manifold 130 to set the position of the proximal end 121 of the microcatheter 122. The distal end 1003 of the adaptor 1000 may be upstream of the proximal end 121 of the microcatheter 122 or optionally may abut the proximal end 121 of the microcatheter 122. It will be appreciated that other arrangements, such as may depend on one or more characteristics of the injectable treatment material delivery system 100 and/or the injectable treatment material, are within the scope and spirit of the present disclosure.

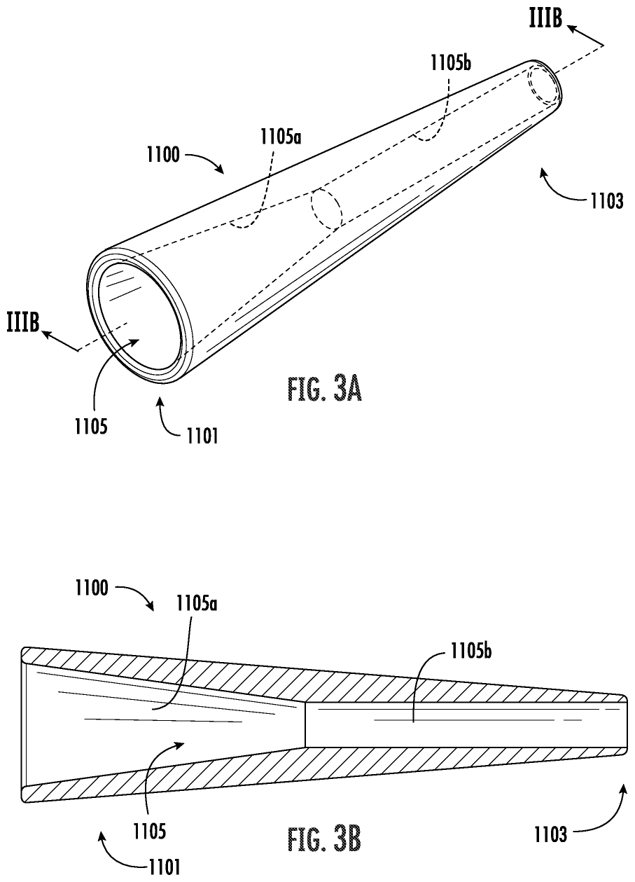

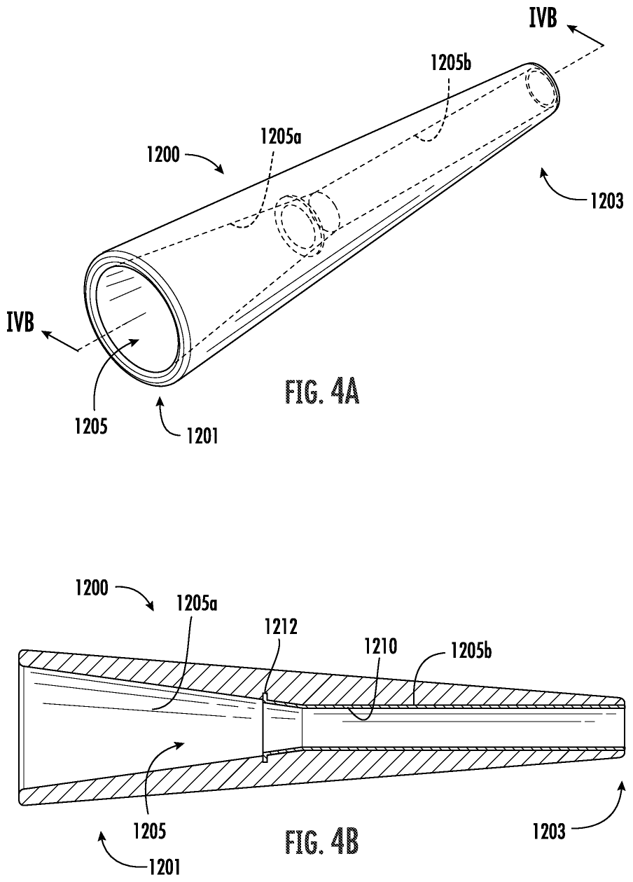

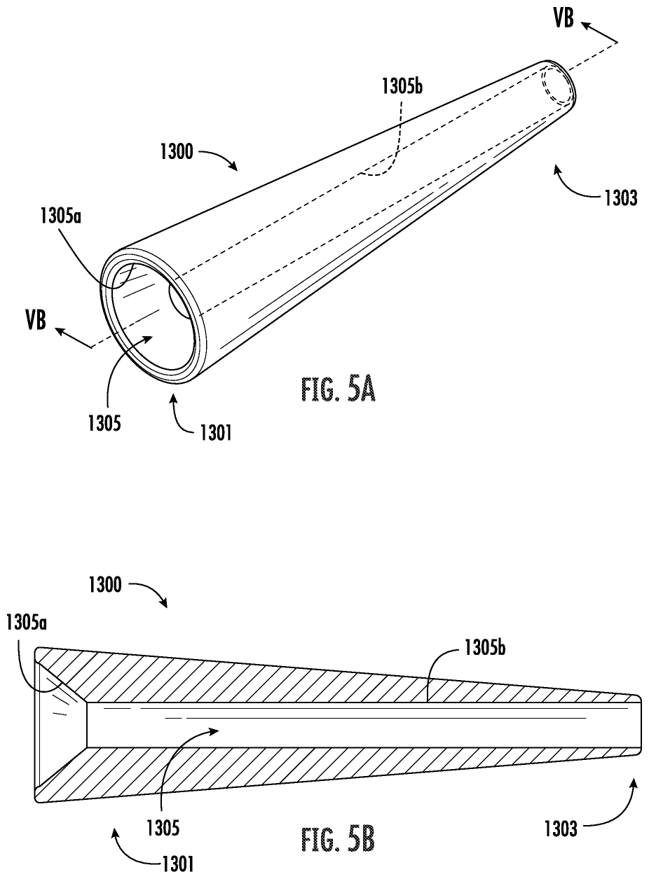

Because an adaptor 1000 formed in accordance with various principles of the present disclosure is formed separately from the injectable treatment material delivery system 100, the material need not be limited by manufacturing processes used in connection with the injectable treatment material delivery system 100. Preferably, however, the material of the adaptor 1000 is selected at least in view of compatibility with the injectable treatment material delivery system 100: The material of an adaptor 1000 formed in accordance with various principles of the present disclosure is moldable, such as a moldable resin. Additionally or alternatively, the material of an adaptor 1000 formed in accordance with various principles of the present disclosure is pliable to conform to and/or to seal with respect to the components with which it is used, yet has sufficient column strength not to collapse under compression in a manner which would undesirably affect flow of materials therethrough. As may be appreciated, the wall of the body of the adaptor 1000 is sized, shaped, configured, and/or dimensioned and the material thereof is sufficiently strong and resistant to collapse so that patency of the adaptor lumen 1005 is maintained and flow of an injectable treatment material such as microspheres is not compromised or impeded. For instance, the length of the adaptor 1000 may be determined based on the length of the manifold lumen 135, such as to ensure that the adaptor 1000 has sufficient space within the manifold lumen 135 so as not to be compressed within the manifold lumen 135 (which may impede flow through the adaptor 1000). Examples of materials which may be used include, without limitation, thermoplastic elastomers, such as polyether block amides (e.g., Pebax® 25d or 35d block copolymers, made up of rigid polyamide blocks and soft polyether blocks, and manufactured by Arkema); thermoplastic polyester elastomers (e.g., Hytrel® block copolymers, made of a hard (crystalline) segment of polybutylene terephthalate and a soft (amorphous) segment based on polyether chemistry, and manufactured by DuPont); thermoplastic polyurethanes (e.g., Pellethane® aromatic polyether and polyesters, manufactured by Lubrizol); thermoplastic vulcanizates (e.g., Santoprene® dynamically vulcanized polymer alloy composed of cured ethylene propylene diene monomer (EPDM) rubber, manufactured by Celanese); DEHP-free PVC's (polyvinyl chlorides free of (Di(2-ethylhexyl) phthalate); etc. Various configurations of examples of embodiments of adaptors 1100, 1200, 1300 formed in accordance with various principles of the present disclosure are illustrated in further detail in FIG. 3A, FIG. 3B, FIG. 4A, FIG. 4B, FIG. 5A, and FIG. 5B.

The lumen of an adaptor formed in accordance with various principles of the present disclosure may have more than one geometrical configuration. For instance, in FIG. 3A and FIG. 3B, the illustrated example of an embodiment of an adaptor 1100 has a lumen 1105 with a proximal tapered segment 1105a, and a distal segment 1105b having a different degree of taper than that of the proximal tapered segment 1105a. For instance, the distal segment 1105b may be tapered to a lesser extent than the proximal tapered segment 1105a. As illustrated in FIG. 3A and FIG. 3B, the distal segment 1105b may have a substantially constant inner diameter (i.e., substantially no taper).

Optionally, a tubular element may be inserted into at least a portion of the lumen of an adaptor formed in accordance with various principles of the present disclosure. The tubular element may be configured to aid and/or guide the flow of the injectable treatment material through the adaptor and thus through the injectable treatment material delivery system 100 into the patient's body. Such tubular element may be provided in a constant-diameter adaptor lumen or in an adaptor lumen with a varying diameter. The tubular element may have a constant diameter or may be tapered, such as depending on the characteristics of the injectable treatment material, the adaptor lumen, the material of the tubular element, etc. For instance, a tapered tubular element may be provided in a generally constant-diameter segment of the adaptor lumen to add a taper to the lumen. The tubular element may be formed of a material more rigid than the material from which the adaptor is formed. For instance, a rigid tubular element may provide structural support to a flexible adaptor (such as against collapse, or at least partial collapse, of the adaptor lumen, which would be susceptible to compromising and/or impeding flow of the injectable treatment material through the collapsed region), such as to a segment of an adaptor lumen with a diameter reduced relative to the diameter of another segment of the adaptor lumen. A rigid tubular element may be formed of stainless steel or any other biocompatible metal or other biocompatible material. Optionally, the material of the adaptor body is molded over the tubular element. Optionally, the exterior of the tubular element is configured (e.g., textured, rippled, etc.) to provide a mechanical interfitting with the overmolded adaptor material to reduce and preferably eliminate relative movement therebetween.

An example of an embodiment of an adaptor 1200 formed in accordance with various principles of the present disclosure with a rigid tubular element 1210 provided in at least a portion of a lumen 1205 of the adaptor 1200 is illustrated in FIG. 4A and FIG. 4B. The segment in which the rigid tubular element 1210 is provided is the distal segment 1105b, and has a generally constant diameter. However, the rigid tubular element 1210 may alternatively or additionally be positioned in the proximal tapered segment 1205a of the adaptor lumen 1205, and/or a tapered segment of the adaptor lumen 1205. Optionally, an anchoring flange 1212 may be provided at one or both ends of the rigid tubular element 1210 to facilitate mounting of the rigid tubular element 1210 with respect to the adaptor 1200 such that the rigid tubular element 1210 does not dislodge therefrom.

It will be appreciated that various other modifications may be made to an adaptor such as described herein without departing from the general principles of the present disclosure. The configurations of the adaptor may be varied internally or externally, such as may be determined based on various factors, such as the nature, properties, characteristics, etc., of the injectable treatment material and/or of the injectable treatment material delivery system and components thereof (e.g., the size, shape, configuration, and/or dimensions of the manifold, such as the interior of the manifold) with which the adaptor is to be used. Various properties of an adaptor formed in accordance with various principles of the present disclosure may be varied such as to ensure desired flow of injectable treatment materials, such as to reduce turbulent flow and creating high shear forces on surfaces to minimize, if not eliminate, spaces for the injectable treatment materials (e.g., microspheres) to coagulate. In some instances, the degree of taper and/or length of the lumen of an adaptor formed in accordance with various principles of the present disclosure may be varied. In the example of an embodiment of an adaptor 1300 illustrated in FIG. 5A and FIG. 5B, the proximal segment 1305a of the lumen 1305 defined through the adaptor 1300 is tapered to a greater degree and is shorter in length than the taper and length of the proximal segment 1105a of the example of an embodiment of an adaptor 1100 illustrated in FIG. 3A and FIG. 3B. The longer proximal tapered segment 1105a of the adaptor 1100 of FIG. 3A and FIG. 3B may allow the male portion 114b of the administration set fitting 114 to be inserted into the adaptor lumen 1105 and/or may be compressed and sealed upon tightening the connection between the administration set 110 and the manifold 130. In contrast, the shorter proximal tapered segment 1305a of the adaptor 1300 of FIG. 5A and FIG. 5B may be configured to mate against the face of the administration set fitting 114. The male portion 114b of the administration set fitting 114 may be used to push an adaptor 1000, 1100, 1200, 1300 formed in accordance with various principles of the present disclosure into a separately manufactured manifold 130.

It will be appreciated that all devices, systems, and methods discussed herein are examples of devices, systems, and/or methods implemented in accordance with one or more principles of this disclosure. Various features described with respect to one embodiment may be applied to another embodiment, whether or not explicitly indicated. It will be appreciated that the examples described herein are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Accordingly, the present invention is not limited to only the embodiments specifically described herein. Other examples of manners of implementing the disclosed principles, and various further benefits of the various aspects, features, components, and structures of devices and systems disclosed herein will occur to a person of ordinary skill in the art upon reading this disclosure.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An adaptor configured to modify a separately manufactured delivery system through which an injectable treatment material is delivered to a patient's body, said adaptor comprising an adaptor body extending from a proximal end to a distal end, wherein:
a lumen is defined through said adaptor body, extending between the proximal end and the distal end of said adaptor body;
the adaptor is sized, shaped, configured, and/or dimensioned to fit within a lumen defined through a manifold of the delivery system to reduce the dimensions of the manifold lumen to facilitate flow of the injectable treatment material therethrough from a proximal end of the manifold to a distal end of the manifold;
the proximal end of the adaptor is configured to mate with a male fitting of an administration set configured to deliver an injectable treatment material through the manifold; and
the distal end of the adaptor is configured to be positioned within the manifold lumen upstream of the proximal end of a catheter within the manifold lumen.

2. The adaptor of claim 1, wherein the adaptor lumen includes a tapered segment.

3. The adaptor of claim 2, wherein the tapered segment is adjacent the proximal end of said adaptor body.

4. The adaptor of claim 3, wherein the adaptor lumen has a constant-diameter segment extending from the tapered segment to the distal end of said adaptor body.

5. The adaptor of claim 4, further comprising a tubular element extending within the constant-diameter segment of the adaptor lumen.

6. The adaptor of claim 5, wherein said adaptor body is formed of a pliable material and said tubular element is formed of a material more rigid than the adaptor body material.

7. The adaptor of claim 6, wherein said adaptor body is molded over said rigid tubular element.

8. The adaptor of claim 1, wherein the proximal end of said adaptor is configured to mate against the face of a fitting of an administration set upstream of the manifold of the delivery system, or to accept a male fitting of the administration set therein.

9. An adaptor configured to modify a manifold through which an injectable treatment material flows for delivery to a patient's body, said adaptor comprising an adaptor body extending from a proximal end to a distal end, wherein:
a lumen is defined through said adaptor body, extending between the proximal end and the distal end of said adaptor body;
the adaptor is sized, shaped, configured, and/or dimensioned to be inserted into a lumen defined through the manifold to reduce the dimensions of the manifold lumen to facilitate flow of the injectable treatment material therethrough from a proximal end of the manifold to a distal end of the manifold;
the adaptor is at least partially formed of a pliable material to conform to the manifold lumen to fill space within the manifold lumen; and
the adaptor is sized, shaped, configured, and/or dimensioned and formed of a material so that the adaptor body maintains patency of the adaptor lumen when positioned within a manifold.

10. The adaptor of claim 9, wherein the adaptor lumen includes a tapered segment.

11. The adaptor of claim 10, wherein the tapered segment is adjacent the proximal end of said adaptor body.

12. The adaptor of claim 11, wherein the adaptor lumen has a constant-diameter segment extending from the tapered segment to the distal end of said adaptor body.

13. The adaptor of claim 12, further comprising a tubular element extending within the constant-diameter segment of the adaptor lumen.

14. The adaptor of claim 13, wherein said adaptor body is formed of a pliable material and said tubular element is formed of a material more rigid than the adaptor body material.

15. The adaptor of claim 14, wherein said adaptor body is molded over said rigid tubular element.

16. The adaptor of claim 15, further comprising a tubular element extending within the adaptor body lumen.

17. The adaptor of claim 16, wherein said adaptor body is formed of a pliable material and said tubular element is formed of a material more rigid than the adaptor body material.

18. The adaptor of claim 17, wherein said adaptor body is molded over said rigid tubular element.

19. A method of reconfiguring a system for delivering an injectable treatment material, said method comprising:
inserting a pliable adaptor into the lumen of a pre-existing manifold formed separately and independently of the adaptor; and
fitting the adaptor within the manifold lumen to occupy space therein to reduce the volume within the manifold lumen to facilitate flow of the injectable treatment material through the manifold and into a patient's body.

20. The method of claim 19, further comprising providing a tubular element within the adaptor lumen to maintain patency of the adaptor lumen to facilitate flow of the injectable treatment material therethrough.
